(19) United States
(12) Patent Application Publication
(10) Pub. No.: US 2003/0196667 A1
(43) Pub. Date: Oct. 23, 2003

(54) Meconium aspirator

(76) Inventors: Kenneth S. Croteau, Hesperia, CA (US); Philip N. Rogone, Victorville, CA (US)

Correspondence Address: Kenneth S. Croteau, 7245 Jenkins Avenue, Hesperia, CA, 92345, US

(21) Appl. No.: 10/417,621

(22) Filed: Apr. 17, 2003

Related U.S. Application Data
(60) Provisional application No. 60/373,403, filed on Apr. 18, 2002.

(51) Int. Cl. A61M 16/00

(52) U.S. Cl. 128/207.14

(57) ABSTRACT

An apparatus and associated method for using a meconium removal apparatus, including an endo-tracheal (ET) tube operably coupled to a meconium mover operably coupled to a suction tube. The meconium mover has a sloped portion having an inlet defined thereon. The method includes inserting one end of the ET tube into an infant's airway; applying suction to the meconium aspirator to draw air into the meconium mover through the side inlet, and controllably closing the inlet to increase suction application to controllably draw meconium fluid in the infant's airway into the ET tube, while withdrawing the ET tube from the infant's airway.

This application claims the benefit and priority of U.S. Provisional Application No. 60/373,403, filed Apr. 18, 2002, which is herein incorporated by reference for all purposes.

### BACKGROUND

1. Field of Invention

This invention relates generally to an apparatus and associated method for the removal of meconium from infants.

2. Related Art

In the care of newborns, the immediate removal of meconium from the newborn infant's airway and lungs can mean the difference between a healthy newborn infant and a newborn infant replete with infection, lung damage, and breathing difficulty.

The removal of meconium from the newborn infant's air passageway is a time critical procedure, since as soon as the newborn becomes stressed and cries, meconium may be aspirated into the newborn infant's airway and lungs. To avoid this occurrence the meconium must be removed as soon as possible after birth.

Unfortunately, using common meconium aspirators wastes critical time by requiring that a crucial connecting step between the endo-tracheal (ET) tube and meconium aspirator occur after the ET tube has been inserted into the newborn infant's airway and withdrawal of a guidewire or stylet has occurred.

Since, the physician or clinician cannot remove the guidewire and connect the aspirator alone without wasting even more critical time; the use of most common meconium aspirators requires the services of two medical professionals.

What is needed therefore is a meconium aspirator that removes the need for the services of more than one medical professional and which saves critical time in the process.

### SUMMARY

The present invention provides an apparatus and associated method for removing meconium from the air passageway and lungs of a newborn infant.

The meconium aspirator of the present invention includes an endo-tracheal (ET) tube, a stylet, a meconium mover and a suction tube. The meconium mover includes a strategically positioned side inlet. The side inlet allows for easy removal of a guidewire after placement of the ET tube and provides a means to regulate suction.

Advantageously, the ET tube, the stylet, the meconium mover and the suction tube of the present invention are operably coupled together to form the meconium aspirator prior to the insertion of the ET tube into the infant's airway. A guidewire used to place the ET tube in the newborn's airway can be removed from the meconium aspirator through the strategically placed side inlet, even while the meconium mover and suction tube are coupled to the ET tube.

Even while the guide wire is being removed, suction can be applied to the suction tube to draw air into the suction tube through the side inlet. Once the guidewire is removed, the strategically placed side inlet allows a clinician to control suction by closing the side inlet to increase the suction and controllably draw meconium fluid from the infant's airway and lungs into the suction tube for withdrawal, while at the same time withdrawing the ET tube from the infant's airway.

In one aspect of the present invention, an apparatus is provided for removal of meconium from an infant's airway. The apparatus includes an aspirator having an ET tube operably coupled to a meconium mover with or without an indwelling stylet operably coupled to a suction tube. The meconium mover defines an inlet, which is formed at an angle to a central lumen of the meconium mover.

In yet another aspect of the invention, a method is provided for using a meconium removal apparatus. The method includes providing a meconium aspirator including an endo-tracheal (ET) tube operably coupled to a meconium mover with or without an indwelling stylet operably coupled to a suction tube. The meconium mover has a sloped portion having an inlet defined thereon. The method also includes inserting one end of the ET tube into an infant's airway; applying suction to the meconium aspirator to draw air into the meconium mover through the side inlet, and controllably closing the inlet to increase suction application to controllably draw meconium fluid in the infant's airway into the ET tube, while withdrawing the ET tube from the infant's airway.

These and other features and advantages of the present invention will be more readily apparent from the detailed description of the preferred embodiments set forth below taken in conjunction with the accompanying drawings.

### BRIEF DESCRIPTION OF THE DRAWINGS

FIGS. 1A and 1B are simplified side views of the meconium aspirator of the present invention; and

FIGS. 2A-2C are simplified illustrations of the use of the meconium aspirator with a newborn infant in accordance with the principles of the present invention.

### DETAILED DESCRIPTION

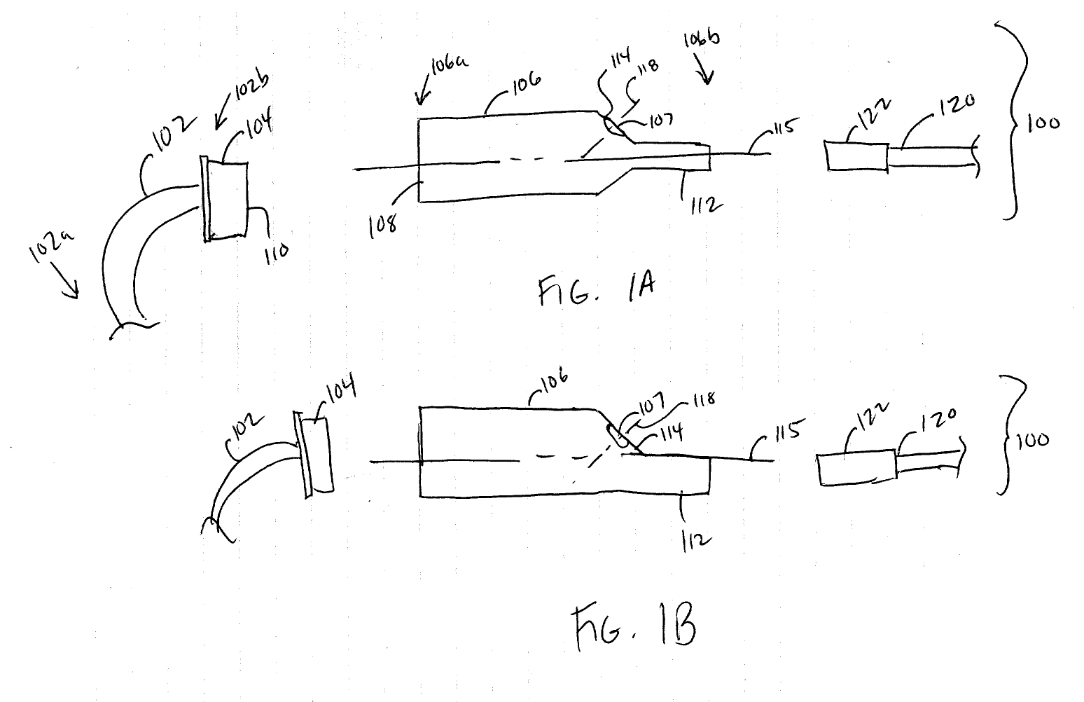

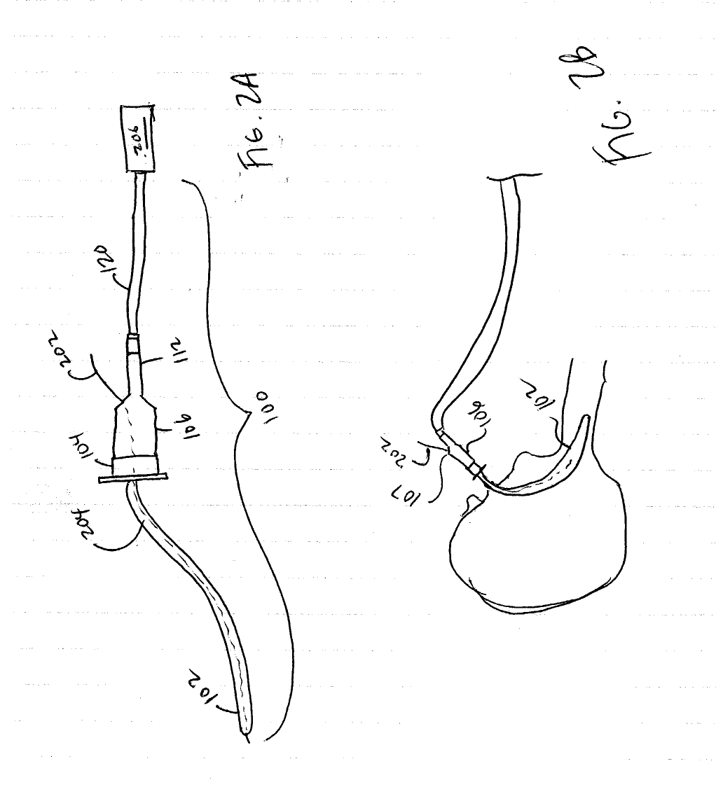

FIGS. 1A and 1B are simplified side views of a meconium aspirator 100 in accordance with the principles of the present invention. Meconium aspirator 100 is used for meconium removal from an infant's airway and lungs. Meconium aspirator 100 includes an endo-tracheal (ET) tube 102, a meconium mover 106 and a suction tube 120. Endo-tracheal tube 102 has a distal end 102a for insertion into an airway of an infant and a proximal end 102b disposed outside the airway.

An adaptor 104 is typically formed on proximal end 102b of ET tube 102 to facilitate the coupling of various aspirator type devices thereto. In this embodiment, adaptor 104 is a typically sized and shaped adaptor for operably receiving meconium mover 106 and coupling it to ET tube 102. Adaptor 104 also provides a means for grasping ET tube 102 so as to pull and remove ET tube 102 from its inserted position in the infant's airway, by manually gripping adapter 104 and pulling adapter 104 and ET tube 102 away from the infant patient.

In one embodiment, meconium mover 106 is a tubular shaped device of between about 6 cm to about 9 cm in length. Meconium mover 106 includes an inlet end 106a, having a first diameter, a sloped portion 114, and an outlet end 106b, having a second diameter. Meconium mover 106 also includes a strategically positioned side inlet 107 also referred to as a stylet hole, which provides a passageway to the internal lumen 108 of meconium mover 106.

Typically a connection between ET tube proximal end 102b and inlet end 106a of meconium mover 106 is made through adapter 104. Generally, inlet end 106a is force fit into a receiving portion 110 of adapter 104. In one embodiment, the inner diameter of inlet end 106a may range from between about 10 mm to about 15 mm, while the outside diameter may range from between about 11.5 and about 16.5 mm.

Outlet end 106b of meconium mover 106 can be defined by a reduced diameter portion 112. Outlet end 106b may have an inner diameter which ranges from between about 4.5 mm to about 6.5 mm and an outer diameter which ranges from between 6 mm to about 8 mm. In one embodiment, reduced diameter portion 112 may include barbs or other similar means for allowing plastic tubing to be removably attached thereto.

Sloped portion 114 provides a transition between inlet end 106a and the reduced diameter portion 112. In one embodiment, as shown in FIG. 1A, sloped portion 114 may be symmetrically formed around centerline 115 of meconium mover 106 to provide a consistently sloped face leading into reduced diameter portion 112. As shown in FIG. 1B, in an alternative embodiment, sloped portion 114 may be formed of a single sloped face offset from centerline 115 that provides tapering of only one side of the cylindrical tube shaped meconium mover 106 leading to reduced diameter portion 112. Sloped portion 114 can be made to have a sloped face of between about 10° to about 90° from centerline 115.

Side inlet 107 can be defined as a hole having a centerline 118 and having any suitable diameter, such as a diameter of up to 10 mm, preferably about 7 mm. In one embodiment, side inlet 107 is formed on sloped portion 114, such that hole centerline 118 forms an angle with centerline 115 of less than 90 degrees, preferably about 45 degrees or less.

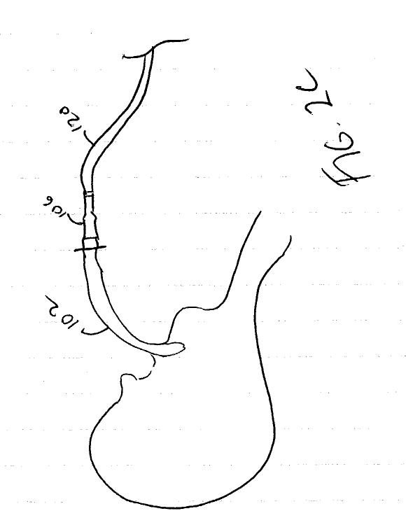

The placement of side inlet 107 at an angle less than 90° with centerline 115 removes the abrupt transition created by a hole placed at a 90° angle with centerline 115. The angled placement of side inlet 107 is advantageous for many reasons. For example, the angled placement of side inlet 107 allows for removal of an indwelling guidewire or stylet 202 (FIG. 2A) through meconium mover 106. This is beneficial in that ET tube 102, meconium mover 106, stylet 202 and suction tube 120 can be coupled together prior to the placement of ET tube 102 into the infant's airway, since the guidewire can be removed through meconium mover 106. In contrast, most other meconium aspirators require placement of the ET tube alone, removal of the guidewire, then coupling of the aspirator to the ET tube.

The angled position of side inlet 107 also provides a better ergonomic position for allowing the clinician the ability to properly grasp meconium aspirator 100 and simultaneously cover side inlet 107 with a finger or thumb and thus control the suction through ET tube 102.

Suction tube 120 can be coupled to reduced diameter portion 112 in any well-known way, such as by means of a rubber or vinyl fitting 122 that can be force fit over reduced diameter portion 112. Suction tube 120 is coupled to a source of regulated suction (not shown). In some embodiments, the amount of suction can range from between about 60 mm and about 90 mm of mercury, preferably about 80 mm of mercury.

When connected, air is drawn into meconium mover 106 via side inlet 107, and flows to suction tube 120. At the appropriate moment, the clinician's finger or thumb can be extended over side inlet 107 to control the amount of air flowing to suction tube 120. Once covered, suction effects are then realized in the infant's airway and lungs. In this way, the clinician is able to control aspiration of the meconium fluid from the airway, via ET tube 102 and thus, avoid the full force of suction to the infant's airway and lungs.

Each of the components of meconium aspirator 100 may be made of any material suitable for use in the clinical applications of aspirators, such as vinyl plastic and the like. Although some dimensions are disclosed, it should be understood that these dimensions are meant to provide examples and are therefore in no way intended to be limiting.

FIGS. 2A-2C provide simplified illustrations for using meconium aspirator 100 in accordance with principles of the present invention. As shown in FIG. 2A, prior to use meconium aspirator 100 is fully assembled by a clinician by coupling together each of the components, including ET tube 102, meconium mover 106 and suction tube 120. Guidewire 202 is placed within the inner lumen 204 defined by the coupled components. Suction tube 120 is coupled to a suction device 206.

As shown in FIG. 2B, after birth, if it us determined that meconium aspiration is necessary, ET tube 120 of meconium aspirator 100 is inserted into the airway of the newly born infant. The clinician grasps meconium mover 106 with one hand, such that the clinician can remove guidewire 202 with the other hand. Once guidewire 202 is removed through side inlet 107, the clinician can control suction by covering side inlet 107 with a finger or thumb.

Once suction is applied to the infant's airway by covering side inlet 107 with a finger or thumb, the clinician simultaneously removes meconium aspirator 100 by pulling ET tube 102 from the infant's airway as shown in FIG. 2C.

Since the clinician did not have to use up critical time between inserting ET tube 102 and removing guidewire 202 to couple meconium mover 106 to adaptor 104 of ET tube 102, a more timely removal of meconium can be achieved.

Having thus described embodiments of the present invention, persons skilled in the art will recognize that changes may be made in form and detail without departing from the scope of the invention. Thus the invention is limited only by the following claims.

What is claimed is:

1. The method of using a meconium removal apparatus, comprising:
providing a meconium aspirator including an endo-tracheal (ET) tube operably coupled to a meconium mover operably coupled to a suction tube, said meconium mover having a sloped portion having an inlet defined thereon;
inserting one end of the ET tube into an infant's airway;
applying suction to the meconium aspirator to draw air into the meconium mover through said side inlet, and
controllably closing said inlet to increase suction application to controllably draw meconium fluid in said airway into the ET tube, while withdrawing said ET tube from the infant's airway.

2. The method of claim 1, wherein said sloped portion is at an angle relative to a centerline of said meconium mover to position said inlet at an angle relative to said centerline.

3. The method of claim 2, wherein said angle comprises an angle of less than 90 degrees.

4. The method of claim 1, wherein said providing a meconium aspirator comprises coupling said ET tube operably to said meconium mover and coupling said meconium mover operably to said suction tube.

5. The method of claim 1, wherein said ET tube and meconium mover include an indwelling stylet.

6. The method of claim 5, further comprising removing said stylet from a position within said ET tube through said inlet.

7. The method of using a meconium removal apparatus, comprising:
coupling together an endo-tracheal (ET) tube, a meconium mover, and a suction tube, said meconium mover having a hole defined on a surface of said meconium mover, said hole providing an angled passageway to a central lumen of said meconium mover; thereafter
inserting one end of the ET tube into an infant's airway;
removing a guidewire through said hole;
applying suction to draw air into the meconium mover through an inlet defined on said meconium mover; and
controllably closing said inlet to increase suction application to controllably draw meconium fluid in said airway into the ET tube, while withdrawing said ET tube from the infant's airway.

8. The method of claim 7, wherein said angle comprises an angle of less than 90 degrees.

9. An apparatus for removal of meconium form an infant's airway comprising:
an aspirator including:
an endo-tracheal (ET) tube operably coupled to a meconium mover operably coupled to a suction tube, said meconium mover defining an inlet, said inlet providing an angled passageway to a central lumen of said meconium mover.
